(19) United States
(12) Patent Application Publication
(10) Pub. No.: US 2010/0049301 A1
(43) Pub. Date: Feb. 25, 2010

(54) Lattice Part Made of Metal and Method for the Production of a Lattice Part

(75) Inventors: Rudolf Zauner, Baden (AT); Franz Riemelmoser, Simbach (DE); Manfred Bammer, Wien (AT)

Correspondence Address: LERNER GREENBERG STEMER LLP, P O BOX 2480, HOLLYWOOD, FL, 33022-2480, US

(73) Assignee: AUSTRIAN RESEARCH CENTERS GMBH - ARC, Wien (AT)

(21) Appl. No.: 12/527,781

(22) PCT Filed: Feb. 12, 2008
(86) PCT No.: PCT/AT2008/000048

§ 371 Date: Nov. 5, 2009

(30) Foreign Application Priority Data
Feb. 19, 2007 (AT) A 259/2007

(51) Int. Cl. A61F 2/82; B32B 3/12; B32B 15/02; B22C 9/00; B32B 1/08

(52) U.S. Cl. 623/1.15; 428/593; 428/586; 164/6; 164/37

(57) ABSTRACT

We describe a lattice part made of metal and a method for producing a lattice part made of metal or a metal alloy. The lattice has a thickness of less than 1 mm at a size of the gaps of less than 50 mm². The lattice comprises a connection made of knots, and perpendicular to the lattice surface has lattice bars and lattice knots of the same thickness. A method for the production of a lattice part made of metal, particularly made of light alloy, is characterized by a primary shaping process according to DIN 8580, wherein a mold is formed in the first step, a primary material is introduced in the mold cavity in the second step, the part is removed from the mold in the third step, and the finishing of the metallic lattice part is carried out in the fourth step.

The invention relates to a lattice part made of metal and to its use.

Furthermore, the invention encompasses a method for the production of a lattice part made of metal or of a metal alloy comprising a metallic node bond of the lattice members, the lattice having a thickness of less than 1 mm with a magnitude of the opening surfaces of less than 50 mm².

Coarse lattice parts made of metal, which have large opening surfaces and a considerable thickness of the lattice members are, in most cases, produced by casting a liquid metal into molds, e.g. into sand molds, by open sand casting or mold casting. It is also known to cut out the opening surfaces from a thick metal sheet, which has to be associated to the prior art for providing heavy lattices.

For the production of fine, planar metallic lattice parts, casting methods, in most cases, cannot be applied, because quality deficiencies in castings, such as material discontinuities, lack of formation of nodes and the like, are often inevitable. For these reasons, lattices, which should have thin members and small opening surfaces, in most cases are produced as a wire mesh.

A fine wire mesh with small opening surfaces has mostly equally shaped narrow mesh widths, because the thickness of the wires in the mesh determine and fix the mutual distance with alternately positioning them at the surface.

In diagonal direction of a lattice or under an angle of about 45°, a wire lattice part, and particularly without fixing of the ends of the parts, may easily be displaced and returned within large limits, because no substantial plastic deformation of the material of the lattice wires is necessary and takes place.

In order to stabilize fine lattices, even with a stress by forces in diagonal direction relative to the layers of wires, one has already attempted to interconnect or bond the places of contact of the lattice members at the nodes by welding or by soldering. Although such an interconnection of the lattice nodes is, in principle, possible, it requires, however, additional expenses.

Moreover, lightweight lattices having a thin wall thickness and an enlarged mesh size and opening surfaces entrain problems with respect to stability of the parts, to a uniform and equal spacing of the members or wires from one another and to a uniform dimension of intermediate surfaces.

Wire lattice parts having a low weight are and with a required fineness of their geometrical shape, particularly with a small ratio of the thickness of the members to the open mesh width, therefore, are inapt to reach the properties in use for a desired profile of requirements.

In this respect, the invention is intended to eliminate the shortcomings, and it has the object to provide a lattice part made of metal, which has a great freedom regarding its shaping as well as the creation of the properties in use, and which does substantially not permit a large diagonal displacement of the lattice that can be formed back, but is plastically deformable and has an accurate, fine dimensioning.

A further object of the invention is to specify a method for the production of a lattice part made of metal of the type described at the outset, by which the part has a desired profile of properties, preferably for the use as a stent or as a stent-like system and as an endoprostesis for the use in the human medical and veterinary medical field.

This object is achieved according to the invention in that the lattice comprises a node bond, consists of metal or of a metal alloy, has a thickness of less than 1 mm with a magnitude of the opening surfaces of less than 50 mm² and that the lattice members and the lattice nodes have the same thicknesses perpendicularly to the lattice surface.

The advantages achieved by the invention consist in particular in that a node bond of the lattice confers to it an improved stability in diagonal direction with respect to the members, and with a formation of light metal results in light weights of the parts. In an advantageous manner, such planar lattices of a small thickness with desired large opening surfaces may present mechanical properties, by which the possibility of use for new applications having a special profile of requirements is made possible.

In one embodiment, the lattice consists of a basis alloy of magnesium, which contains in percent by weight:

| | |
|---|---|
| zinc (Zn) | 0.6 to 8.0, preferably 0.8 to 6.2 |
| zirconium (Zr) | up to 2.0, preferably up to 1.0 |
| manganese (Mn) | 0.02 to 0.8, preferably 0.04 to 0.6 |
| calcium (Ca) | up to 1.2, preferably up to 1.0 |
| antimony (Sb) | up to 0.8, preferably up to 0.5 |
| aluminum (Al) | up to 0.8, preferably up to 0.5 |
| silver (Ag) | 0.8 to 2.5, preferably 0.1 to 2.0 |

and impurities comprising magnesium as the balance.

In accordance with the invention, an application in vivo of lattice parts within and on the body of men and animals is thereby favorable.

Geometrically, but also in view of a stable expansion of the lattice part, optionally locally over the axis, it may be favorable, if the part has a tubular shape, particularly an undivided tubular shape. In such a manner, high stability of the part is achieved with a small weight of the part.

If the lattice, in a preferred embodiment according to the invention, is produced by casting metal in accordance with DIN 8580, any diagonal displacement of the lattice members can be prevented without any lasting deformation at the nodes, thus favorizing lasting shaping of the lattice surface. In such a manner, threedimensional lattice surfaces, e.g. partially expanded lattice tubes, may be produced in accordance with the desired profile of properties.

Furthermore, it is particularly advantageous for tubular lattice parts, if the lattice members have an angle unequal to 90° or unequal to 0° in relation to the tube axis, and have in particular an angle of about 45°. In such a manner, when expanding the lattice members, the lattice members are torsion-deformed, substantially in a lasting manner so that stretching or an elongation of members, and thus the risk of breakage, are minimized.

If at least one of the lattice surfaces is machined, a very small lattice thickness can be produced, even with casting a metal lattice, so that economic, process engineering advantages are obtained and also in view of quality, and the lattice properties have likewise a desired stability.

Moreover, a fine lattice tube has advantageously a wall thickness of more than 0.1 mm, but smaller than 0.4 mm, and a diameter of more than 1 mm, but smaller than 4 mm. For such a lattice, important opportunities are given in a technical field of application, but also in the medical field, particularly for the endoprosthetic care of men and animals.

If the lattice, as according to a preferred embodiment, is plastically deformable, in particular if a lattice tube can be expanded in diameter in a lasting way, and if the lattice material consists of a basis alloy of magnesium, as mentioned above, the part may be used in a favorable manner as a stent or as a stent-like system and as an endoprostesis for the use in the human medical and veterinary medical field, particularly in blood vessels of men and animals. In this case, body liquids, slowly dissolve at least the surface region of the metallic stent, while forming calcium hydroxyl apatite, a cartilaginous mass, which is able to take over excellent supporting functions.

The further object of the invention to provide a method for the production of a lattice part made of a light metal or of a light metal alloy comprising a metallic node bond of the lattice members, the lattice having a thickness of less than 1 mm with a magnitude of the opening surfaces of less than 50 mm² is achieved in that the lattice is produced by casting according to DIN 8680, wherein

in a first step at least a part of the mold is formed, after which, after jointing the mold,

in a second step a starting material comprising metal or a like alloy at least in part is introduced into the cavity of the mold or is applied to a profiled mold part, and a master pattern is established, after which

in a third step the part is at least partially removed from the mold, and

in a fourth, subsequent step finishing of the metallic lattice part is effected.

There is a variety of advantages of the method according to the invention, and shall be seen or reside in the achievement of favorable lattice properties in an advantageous manner of production and in a high efficiency. Casting the fine lattice of metal insures a desired stability of the lattice nodes as well as the properties, which are preferred in use. In this particular case of casting, it has shown to be advantageous, if in a first step at least part of the mold is formed, because in this way high flexibility of the production method is reached. After jointing the mold, which comprises also supporting it for introducing the metal under pressure, in a second step introducing the starting material in to the cavity of the mold, wherein a flowable metal, which includes solidified particles, may advantageously used.

According to another variant of the method, metal may be applied onto a mold part, manufactured as has previously been explained, in accordance with the liquid or semi-liquid metal spray method and can be solidified. In the subsequent third step, the part can optionally be removed from the mold or can be released from the mold in part only, one mold part having a supporting function, e.g. for machining the lattice. In the last step, finishing of the metallic lattice part is effected, which ensures the intended shape for use and quality.

If in the first step of the production method, at least part of the mold is formed by Powder Injection Molding (PIM), a particularly exact mold shaping can be achieved.

When doing this, it may be favorable, if that mold part, which has been produced in the first step by Powder Injection Molding (PIM), is produced making a mold material by mixing powder, such as ceramic powder, and a binding agent, granulating the mold material and injection molding the granules into a mold, thus forming a green body which can be further made into a brown body, which by sintering can be made to a mold part, preferably having a porosity of 75 to 95% by volume.

In such a way, it is not only the strength of the mold or the mold material which is influenced in a favorable manner, but mold release of a molded part is promoted too.

In view of a particularly high quality of the lattice members, and in particular of the lattice nodes, but also in view of weight and usefulness, it has shown to be advantageous, if a basis alloy of magnesium is used as a light metal, which contains in percent by weight:

| | |
|---|---|
| zinc (Zn) | 0.6 to 8.0, preferably 0.8 to 6.2 |
| zirconium (Zr) | up to 2.0, preferably up to 1.0 |
| manganese (Mn) | 0.02 to 0.8, preferably 0.04 to 0.6 |
| calcium (Ca) | up to 1.2, preferably up to 1.0 |
| antimony (Sb) | up to 0.8, preferably up to 0.5 |
| aluminum (Al) | up to 0.8, preferably up to 0.5 |
| silver (Ag) | 0.8 to 2.5, preferably 0.1 to 2.0 |

and impurities comprising magnesium as the balance.

If, in the second step, at least partially liquid metal is introduced into the mold cavity, particularly by die casting, shrinkage can be reduced in a favorable manner during solidification of the metal, and the micro-structure of the lattice part can advantageously be made finer.

It is also possible, and it can be advantageous for certain types of production of a lattice part, if, in the second step, a starting material of metal powder and of a binding agent is introduced in to the mold cavity, particularly by injection molding or by Metal Injection Molding (MIM), while in the fourth step of the production, finishing is effected by densesintering.

For producing particularly fine and thin lattice parts, which do not exhibit sufficient shaping strength to be machined, the part, in the third step, may be partially released out of or from the mold in such a manner, that it is left on a portion of the mold, thus being supported by it, after which, in the fourth step, finishing of the metallic lattice part is effected by machining for producing the desired exact dimensions, and by subsequent removal of the supporting mold portion, particularly by washing it out or by chemically dissolving it.

Subsequently, a possible production of a lattice part according to the invention shall be explained in accordance with the invention, wherein graphical plots may serve as an aid.

It is shown in

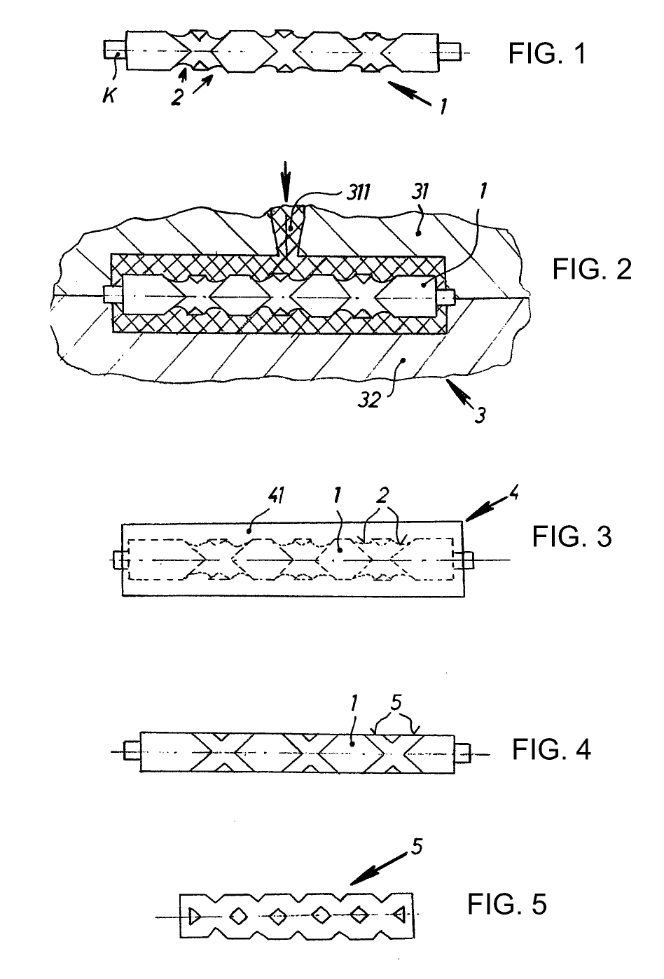

FIG. 1 a core part of a mold

FIG. 2 a mold comprising a core part

FIG. 3 the mold after casting

FIG. 4 a lattice part on a mold core

FIG. 5 a lattice part

By Powder Injection Molding (PIM), a core part 1 according to FIG. 1 is produced and is sintered and includes core marks K for holding it in a mold, as well as lattice-like recesses 2.

FIG. 2 shows a core part 1 when inserted into an injection mold 3 that is formed by a upper part 31 including a melt supply channel 311, and a lower part 32, a mold cavity being formed between the core part 1 and the mold 3.

FIG. 3 illustrates a cast body 4 surrounding the core part and solidified within the cavity of the mold, after the cast body 4 has been released from the injection mold 3. Lattice forming recesses 2 on the core part 1 are filled, and, if necessary, a cylindrical outer part 41 is provided on the cast body 4 and serves optionally for providing starting material for a lattice on all sides.

After machining the outer surface of the cast body 4 on the supporting core part 1, a lattice part 5 is produced on it, as is represented in FIG. 4.

As shown in FIG. 5, a lattice part 5 is obtained after dissolving chemically the core part 1, and optionally after a further treatment for an intended use.

1-16. (canceled)

17. A lattice part made of metal, comprising:
a lattice formed of a node bond with lattice members and lattice nodes, and consisting of metal or of a metal alloy;
said lattice having a thickness of less than 1 mm and openings with a surface area of less than 50 mm²; and
said lattice members and said lattice nodes having a common thickness perpendicularly to a lattice surface.

18. The lattice part according to claim 17, wherein said lattice consists of a base alloy of magnesium containing, in percent by weight:

| | |
|---|---|
| zinc (Zn) | 0.6 to 8.0; |
| zirconium (Zr) | up to 2.0; |
| manganese (Mn) | 0.02 to 0.8; |
| calcium (Ca) | up to 1.2; |
| antimony (Sb) | up to 0.8; |
| aluminum (Al) | up to 0.8; |
| silver (Ag) | 0.8 to 2.5; |

impurities, and remainder magnesium.

19. The lattice part according to claim 18, wherein said base alloy contains, in percent by weight:

| | |
|---|---|
| zinc (Zn) | 0.8 to 6.2; |
| zirconium (Zr) | up to 1.0; |
| manganese (Mn) | 0.04 to 0.6; |
| calcium (Ca) | up to 1.0; |
| antimony (Sb) | up to 0.5; |
| aluminum (Al) | up to 0.5; |
| silver (Ag) | 0.1 to 2.0. |

20. The lattice part according to claim 17, wherein the part has a tubular shape.

21. The lattice part according to claim 17, formed into a part with an undivided tubular shape.

22. The lattice part according to claim 17, wherein said lattice is a cast metal part produced in accordance with DIN 8580.

23. The lattice part according to claim 17, wherein said lattice is formed in a tubular shape with a tube axis and said lattice members have an angle unequal to 90° or unequal to 0° in relation to said tube axis.

24. The lattice part according to claim 23, wherein said lattice members enclose an angle of substantially 45° with said tube axis.

25. The lattice part according to claim 17, wherein at least one lattice surface is a machined lattice surface.

26. The lattice part according to claim 17, wherein said lattice is a lattice tube with a wall thickness of more than 0.1 mm and less than 0.4 mm, and a diameter of more than 1 mm and less than 4 mm.

27. The lattice part according to claim 17, wherein said lattice is a plastically deformable lattice.

28. The lattice part according to claim 17, wherein said lattice is a lattice tube with a non-elastically expandable diameter.

29. The lattice part according to claim 17, formed as a stent or as a stent-like system and as an endoprostesis for use in human medical and veterinary medical fields.

30. A method for producing a lattice part made of metal or of a metal alloy and forming a metallic node bond of lattice members, with the lattice having a thickness of less than 1 mm and openings with a surface area of less than 50 mm², the method which comprises:
producing the lattice by casting according to DIN 8680 and
in a first step, forming at least a part of the mold and jointing the mold;
in a second step, introducing a starting material of metal or a metal alloy at least in part into a cavity of the mold or applying the starting material to a profiled mold part, and establishing a master pattern;
subsequently, in a third step, at least partially removing the molded part from the mold; and
subsequently, in a fourth step, finishing of the molded part into a metallic lattice part.

31. The method according to claim 30, wherein the first step comprises forming at least part of the mold by powder injection molding (PIM).

32. The method according to claim 30, wherein in the first step, that mold part, which has been produced in the first step by powder injection molding (PIM), is produced making a mold material by mixing powder and a binder, granulating the mold material and injection molding the granules into a mold, thus forming a green body which can be further made into a brown body, which by sintering can be made to a mold part.

33. The method according to claim 32, which comprises sintering the brown body into a sintered body having a porosity of 75 to 95% by volume.

34. The method according to claim 32, which comprises mixing ceramic powder with the binder.

35. The method according to claim 30, which comprises using a base alloy of magnesium containing, in percent by weight:

| | |
|---|---|
| zinc (Zn) | 0.6 to 8.0; |
| zirconium (Zr) | up to 2.0; |
| manganese (Mn) | 0.02 to 0.8; |
| calcium (Ca) | up to 1.2; |
| antimony (Sb) | up to 0.8; |
| aluminum (Al) | up to 0.8; |
| silver (Ag) | 0.8 to 2.5; |

impurities, and remainder magnesium.

36. The method according to claim 35, wherein the base alloy contains, in percent by weight:

| | |
|---|---|
| zinc (Zn) | 0.8 to 6.2; |
| zirconium (Zr) | up to 1.0; |
| manganese (Mn) | 0.04 to 0.6; |
| calcium (Ca) | up to 1.0; |
| antimony (Sb) | up to 0.5; |
| aluminum (Al) | up to 0.5; |
| silver (Ag) | 0.1 to 2.0. |

37. The method according to claim 30, wherein the second step comprises introducing at least partially liquid metal into the mold cavity.

38. The method according to claim 37, wherein the second step comprises die casting the metal into the mold cavity.

39. The method according to claim 30, wherein the second step comprises introducing a starting material of metal powder and a binder into the mold cavity, and the fourth step comprises finishing by dense-sintering.

40. The method according to claim 39, which comprises introducing the starting material into the mold cavity by injection molding or by metal injection molding.

41. The method according to claim 30, wherein the third step comprises partially releasing the part out of or from the mold such that the part remains on a portion of the mold and is supported thereby, and wherein the subsequent fourth step comprises finishing the metallic lattice part by machining for producing desired exact dimensions, and subsequently removing the supporting mold portion.

42. The method according to claim 41, wherein the step of removing the supporting mold portion comprises washing the mold portion out or chemically dissolving the mold portion.
